(19) United States
(12) Patent Application Publication
(10) Pub. No.: US 2008/0299041 A1
(43) Pub. Date: Dec. 4, 2008

(54) HETEROCYCLIC INDENE DERIVATIVES AND THEIR RADIOISOTOPE LABELED COMPOUNDS FOR IMAGING BETA-AMYLOID DEPOSITION

(75) Inventors: Jae Min JEONG, Seoul (KR); Dong Soo LEE, Seoul (KR); June-Key CHUNG, Seoul (KR); Myung Chul LEE, Seoul (KR)

Correspondence Address: HESLIN ROTHENBERG FARLEY & MESITI PC, 5 COLUMBIA CIRCLE, ALBANY, NY, 12203, US

(73) Assignee: SEOUL NATIONAL UNIVERSITY INDUSTRY FOUNDATION, Seoul (KR)

(21) Appl. No.: 12/128,005

(22) Filed: May 28, 2008

(30) Foreign Application Priority Data
Jun. 1, 2007 (KR) 10-2007-0053777

(51) Int. Cl. A61K 51/04; C07D 277/62; C07D 263/54; A61P 25/28; C07D 333/52; C07D 307/87

(52) U.S. Cl. 424/1.89; 548/152; 548/217; 549/49; 549/462

(57) ABSTRACT

The invention is directed to heterocyclic indene derivatives useful for β-amyloid plaque imaging, their radiolabeled compounds and their preparation methods. The compounds of the invention are easily labeled with radioisotopes and have high affinities to β-amyloid depositions, thus they facilitate diagnosis of Alzheimer's disease by imaging the distribution of β-amyloid depositions.

### CROSS-REFERENCE TO RELATED APPLICATIONS

This application claims priority to Korean Patent Application No. 10-2007-0053777, filed on Jun. 1, 2007, which is hereby incorporated herein by reference in its entirety.

### TECHNICAL FIELD

The present invention relates to novel heterocyclic indene derivatives for imaging β-amyloid depositions by binding them and consequent radiation from binding site, their radiolabeled compounds, precursors for synthesizing them, and their preparation method.

### BACKGROUND ART

β-amyloid depositions are found in the brain of Alzheimer's disease patient. Alzheimer's disease is characterized by decrease of brain nerve cells resulting in reduced memory and cognitive power. Plaques or tangles that are formed by aggregation of β-amyloid peptide are found in the Alzheimer's brain. Alzheimer's disease might be suppressed by administration of drugs inhibiting formation of β-amyloid plaques and tangles.

Although Alzheimer's disease can be confirmed by staining the postmortem brain with Congo red, it cannot be applied to alive human. Congo red cannot enter into brain when it is administrated to human body, because it is impermeable to blood-brain-barrier (BBB) due to high hydrophilicity. Thus, in order to image and diagnose Alzheimer's disease it is necessary to radiolabel a BBB-permeable compound that can bind to β-amyloid plaques.

The earliest radiolabeled compounds for imaging β-amyloid plaques were Congo red (1) and Chrysamine-G (2) derivatives as shown in Formula 3. However, these compounds were not practically applicable due to low BBB-permeability (Klunk W E, Debnath M L, Pettegrew J W. Development of small molecule probes for the beta-amyloid protein of Alzheimer's disease. Neurobiol Aging 1994, 15:691-8; and Klunk W E, Debnath M L, Pettegrew J W. Chrysamine-G binding to Alzheimer and control brain: Autopsy study of a new amyloid probe. Neurobiol Aging 1995, 16:541-8).

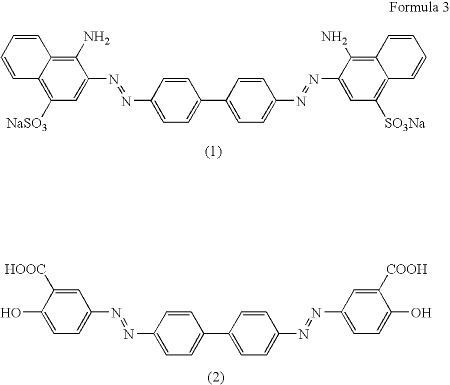

The research became more active after the development of 6-dialkylamino-2-naphthylethylidene (FDDNP) (1) and thioflavin-T (2) derivatives of Formula 4 (Agdeppa E D, Kepe V, Liu J, Flores-Torres S, Satyamurthy N, Petric A, Cole G M, Small G W, Huang S C, Barrio J R. Binding characteristics of radiofluorinated 6-dialkylamino-2-naphthylethylidene derivatives as positron emission tomography imaging probes for β-amyloid plaques in Alzheimer's disease. J Neuroscience 2001, 21:1-5; and Mathis C A, Bacskai B J, Kajdasz S T, MlLellan M E, Frosch M P, Hyman B T, Holt D P, Wang Y, Huang G-F, Debnath M L, Klunk W E. A lipophilic thioflavin-T derivative for positron emission tomography (PET) imaging of amyloid in brain. Bioorg Med Chem Lett 2002, 12:295-298).

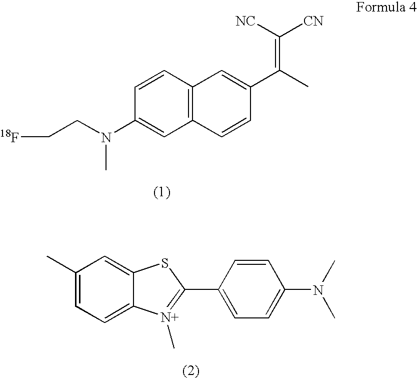

Benzothiazole (1) and stilbene (2) derivatives of Formula 5 were reported as radiolabeled agents for β-amyloid plaque imaging. (US Patent Pub. No. 2002/0133019 A1, W. E. Klunk, C. A. Mathis Jr, Thioflavin derivatives for use in antemortem diagnosis of Alzheimer's disease and vivo imaging and prevention of amyloid diposition; and US Patent Pub. No. 2003/0149250 A1, H. F. Kung, M-P. Kung, Z-P. Zhuang, Stilbene derivatives and their use for binding and imaging amyloid plaques).

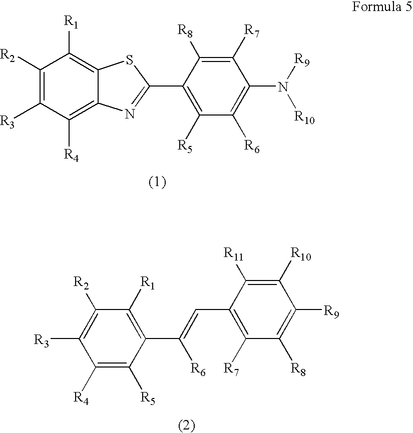

Benzothiazole derivatives developed into various heterocyclic indene derivatives containing nitrogen, sulfur and oxygen atoms, and contributed to β-amyloid imaging.

Benzoxazole derivatives of Formula 6 showed high binding affinity to β-amyloid and rapid uptake and excretion. (Zhuang Z-P, Kung M-P, Hou C, Plossl K, Skovronsky D, Gur T L, et al. IBOX (2-(4′-dimethylaminophenyl)-6-iodobenzoxazole): a ligand for imaging amyloid plaques in the brain. Nucl Med Biol 2001; 28:887-94)

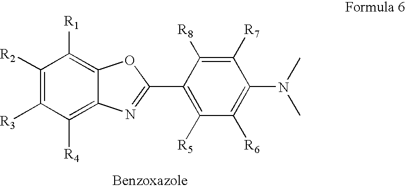

Many benzofuran derivatives of Formula 7 have been synthesized and also showed high affinity to β-amyloid protein. An important point is that the affinity was retained after replacement of amino residue with methoxy or hydroxyl residue. (Ono M, Kung M-P, Hou C, Kung H F. Benzofuran derivatives as Aβ-aggregate-specific imaging agents for Alzheimer's disease. Nucl Med Biol 2002; 29:633-42)

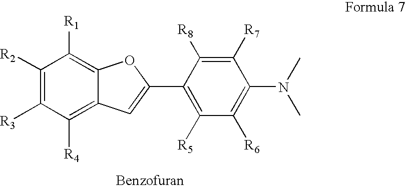

Benzothiophene derivatives of Formula 8 having the same basic structure were developed and showed feasibility as a β-amyloid imaging agent due to high β-amyloid affinity and high brain uptake. (Chang Y S, Jeong J M, Lee Y-S, Kim H W, Rai B G, Kim Y J, et al. Synthesis and evaluation of benzothiophene derivatives as ligands for imaging β-amyloid plaques in Alzheimer's disease. Nucl Med Biol 2006; 33:811-820)

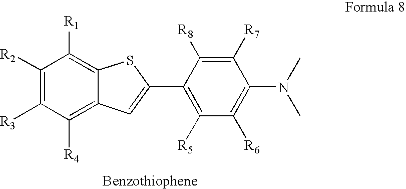

Above reports clarify that if an indene derivative has an aniline, N-methylaniline or N,N-dimethylaniline at 2 position, and a 1 or 3 position carbon is replaced with either sulfur, oxygen or nitrogen, thereof having structure of Formula 9, then they can be used as a β-amyloid imaging agent due to high affinity to β-amyloid.

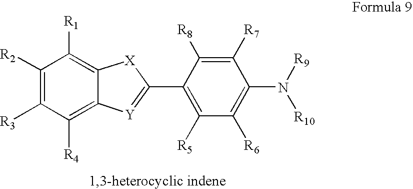

As shown above, it has been reported that all the heterocyclic indene derivatives bound with aniline, phenol or their analogues are useful for diagnosis, imaging and prevention of Alzheimer's disease (Klunk W E, Mathis C A, Wang Y. Thioflavin derivatives for use in antemortem diagnosis of Alzheimer's disease and vivo imaging and prevention of amyloid deposition. US 2002/0133019 A1, Sep. 19, 2002).

### DISCLOSURE OF THE INVENTION

The object of the present invention is to provide novel compounds for imaging β-amyloid deposits and precursors for synthesize them. For this, the compounds should be easy to radiolabel, have high β-amyloid binding affinity, and show fast whole body clearance.

The other object of the present invention is to provide a method for preparation of said compound.

Another object of the present invention is to provide a composition for β-amyloid imaging comprising said compound.

Another object of the present invention is to provide a pharmaceutical composition for diagnosis of Alzheimer's disease.

The present invention provides a heterocyclic indene derivative compound described by the following Formula 1, wherein, X is selected from a group consisting of O, S and NH; Y is CH or N; Z is selected from a group consisting of F, Cl, Br, I and a sulfonyl derivative; and R₁ to R₈ are independently selected from a group consisting of hydrogen, C₁˜C₄ alkyl, F, Cl, Br, I, O—CH₃, O—CH₂—CH₃, O—CH₂—CH₂—CH₃ and OH, respectively.

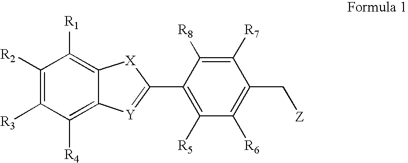

The example of said sulfonyl derivatives includes methansulfonyl (OMs), toluenesulfonyl (OTs), trifluoromehanesulfonyl (OTf), benzenesulfonyl, nitrobenzenesulfonyl, mesitylenesulfonyl, triisopropylbenzenesulfonyl, chlorobenzenesulfonyl, and dichlorobenzenesulfonyl.

Further, it is preferably that the substituents R₁ to R₈ in Formula 1 are all hydrogen, respectively.

The heterocyclic indene derivative compound of the present invention has enough lipophilicity for BBB penetration resulting in high initial brain uptake and rapid brain washout.

Further, the heterocyclic indene derivative compound can be used as precursors for radioisotope labeled compounds of the heterocyclic indene derivative compound.

Another embodiment of the present invention is radiolabeled heterocyclic indene derivatives described as the following Formula 2, wherein X is selected from a group consisting of O, S and NH; Y is CH or N; and R₁ to R₈ are independently selected from a group consisting of hydrogen, C₁˜C₄ alkyl, F, Cl, Br, I, C—CH₃, C—CH₂—CH₃, C—CH₂—CH₂—CH₃ and OH, respectively.

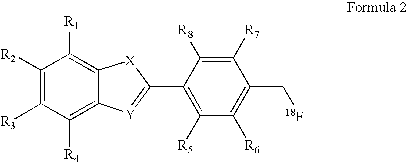

Further, the substituents R₁ to R₈ in Formula 2 are preferably hydrogen, respectively.

The radioisotope labeled compounds of the present invention have high affinity to β-amyloid deposits, thus they can be used as β-amyloid deposit imaging agents.

Another embodiment of the present invention relate to a method for preparation of the radioisotope labeled heterocyclic indene derivatives, which comprises reacting the compound of claim 1 with an activated ¹⁸F.

In the method of the present invention, the activated ¹⁸F is in form of a quaternary ammonium salt or a cation containing crown ether salt.

In the method of the present invention, the quaternary ammonium is tetrabutyl ammonium bicarbonate and the cation containing crown ether is a mixture of K₂CO₃ and Kryptofix 2.2.2.

Other generally known ¹⁸F⁻ activation methods also can be applied.

An example of radiolabeling method of present invention is described in Scheme 1.

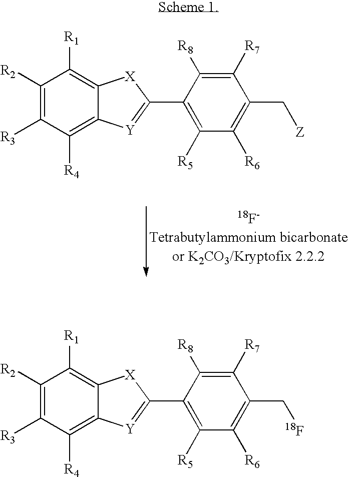

wherein X is selected from a group consisting of O, S and NH; Y is CH or N; and R₁ to R₈ are independently selected from a group consisting of hydrogen, C₁˜C₄ alkyl, F, Cl, Br, I, C—CH₃, C—CH₂—CH₃, C—CH₂—CH₂—CH₃ and OH, respectively.

Above sulfonyl derivatives includes methansulfonyl (OMs), toluenesulfonyl (OTs), trifluoromehanesulfonyl (OTf), benzenesulfonyl, nitrobenzenesulfonyl, mesitylenesulfonyl, triisopropylbenzenesulfonyl, chlorobenzenesulfonyl, and dichlorobenzenesulfonyl.

¹⁸F⁻ can be produced by bombarding ¹⁸O—H₂O with proton beam accelerated by cyclotron. 18F— is in water solution just after production which is inactive form. It is activated by drying with crown ether especially Kryptofix 2.2.2 and K2CO3 mixture or quaternary ammonium especially tetrabutylammonium bicarbonate. To the activated 18F—, a compound of the first embodiment of the present invention, so called a precursor, is added and reacted, and then a labeling reaction occurs. Precursors are the compounds that have strong electron withdrawing group such as halogen or sulfonyl in place of ¹⁸F, so that they can be replaced by activated ¹⁸F⁻.

This generally known radiolabeling method using activated ¹⁸F⁻ is a nuclear substitution method. (Chang Y S, Jeong J M, Lee Y-S, Kim H W, Rai B G, Kim Y J, et al. Synthesis and evaluation of benzothiophene derivatives as ligands for imaging β-amyloid plaques in Alzheimer's disease. Nucl Med Biol 2006; 33:811-820)

The forth embodiment of the present invention comprises the composition for β-amyloid imaging containing radiolabeled heterocyclic indene derivatives. The preferred radioactivity and content of the radiolabeled composition are 0.1 mCi˜10 Ci and 1 ng/administration 100 mg/administration, respectively. The composition preferably is pharmaceutically acceptable non-pyrogenic and sterile formula.

The fifth embodiment of the present invention comprises the pharmaceutical injection composition for diagnosis of Alzheimer's disease containing radiolabeled heterocyclic indene derivatives. The preferred radioactivity and content of the radiolabeled composition are 0.1 mCi˜10 Ci and 1 ng/administration ˜100 mg/administration, respectively. The composition preferably is pharmaceutically acceptable non-pyrogenic and sterile formula.

An important achievement of the present invention is the introduction of benzyl fluoro type ¹⁸F which can be metabolized by liver microsomal enzyme rapidly, thus the clearance from the blood consequently from the brain also is rapid. Unbound compounds are metabolized rapidly in the liver and then taken up by the bone or excreted into urine rapidly. Eventually, better images can be obtained by reduced background radioactivity.

Heterocyclic indene derivatives containing fluorobenzyl or precursors for labeling them have never been reported or patented previously. In addition, they have never been reported or patented as imaging agents for β-amyloid deposits.

The compounds in the present invention can penetrate BBB easily due to lipophilicity and have high affinities to β-amyloid plaques and tangles which are expressed in the brain of Alzheimer's disease, thus can be used for therapy or diagnosis of Alzheimer's disease.

The penetration of BBB can be estimated by animal experiment. The radiolabeled compounds are intravenously injected to mice, and then the brains are obtained at 2 min, 30 min, and 60 min. The radioactivities of the brain are expressed as the percentage of injected dose per brain weight (gram).

Affinity to β-amyloid can be measured by incubation with β-amyloid precipitate and count the bound form after separation by centrifugation. Kd or Ki values are obtained either by saturation binding assay or competition assay.

In addition, the compounds in the present invention can bind with β-amyloid deposits not only in the brain but also in other organs. Thus, they also can be applied for diagnosis and therapy of peripheral deposition of β-amyloid such as amyloidosis.

### BRIEF DESCRIPTION OF THE DRAWINGS

FIG. 1. HPLC chromatogram of 2-(4′-[¹⁸F]fluoromethyl)phenyl-1,3,-benzothiazole in Example 10, wherein, Red line ([¹⁸F]1 represents a radioactivity and green line (Cold 1) shows a UV absorbance.

FIG. 2. Biodistribution graph observed in each organ after injection of 2-(4′-[¹⁸F]fluoromethyl)phenyl-1,3-benzothiazole through the mice tail veins. The mice were sacrificed at 2 min, 30 min and 60 min after injection, respectively, and then organs were taken out. The organs were weighed and their radioactivities were measured. The radioactivity distribution in each organ was calculated as the percentage of injected dose per organ weight (gram).

### BEST MODE FOR CARRYING OUT THE INVENTION

The following examples are given to illustrate the present invention. It should be understood, however, that the invention is not to be limited to the specific conditions or details described in these examples.

### EXAMPLE 1

### Synthesis of 2-(4′-fluoromethyl)phenyl-1,3-benzothiazole

To the solution of 2-aminothiophenol (94 μL, 0.9 mmol) in toluene (5 mL), 4-(fluoromethyl)-1-benzenecarbonyl chloride (150 mg, 0.9 mmol) was added. After refluxing for 20 hrs, the reaction mixture was filtered, and the filtrate volume was reduced by evaporating and recrystallized in EtOAc and toluene mixture to afford 2-(4′-fluoromethyl)phenyl-1,3-benzothiazole (138 mg, 0.6 mmol) as a yellowish solid in a yield of 65%. ¹H NMR (300 MHz, CDCl₃): δ 5.38 (s, 1H, —CH₂—), 5.54 (s, 1H, —CH₂—), 7.41 (m, 1H), 7.50 (m, 3H), 7.92 (d, 1H, J=7.8 Hz), 8.01-8.14 (m, 3H).

### EXAMPLE 2

### Synthesis of 2-(4′-fluoromethyl)phenyl-1,3-benzoxazole

To the solution of 2-aminophenol (95 μL, 0.9 mmol) in toluene (10 mL), 4-(fluoromethyl)-1-benzenecarbonyl chloride (150 mg, 0.9 mmol) was added. After refluxing for 40 hrs, the reaction mixture was filtered, and the filtrate volume was reduced by evaporating. The residue was purified with column chromatography in CH₂Cl₂ and MeOH (30:1) mixture to afford 2-(4′-fluoromethyl)phenyl-1,3-benzoxazole (38 mg, 0.1 mmol) as a yellowish solid in a yield of 15%. ¹H NMR (300 MHz, CDCl₃): δ 5.40 (s, 1H, —CH₂—), 5.56 (s, 1H, —CH₂—), 7.37 (m, 2H), 7.53 (d, 2H, J=7.8 Hz), 7.58 (m, 1H), 7.78 (m, 1H), 8.29 (d, 2H, J=7.8 Hz).

### EXAMPLE 3

### Synthesis of 2-(4′-fluoromethyl)phenylbenzo[b]thiophene

To the solution of (2-sulfanylphenyl)methyltriphenylphosphonim bromide (493 mg, 1 mmol) in toluene (10 mL), 4-(fluoromethyl)-1-benzenecarbonyl chloride (123 mg, 1.0 mmol) and Et₃N (302 μL, 2.3 mmol) were added. After refluxing for 20 hrs, the reaction mixture was filtered, and the filtrate volume was reduced by evaporating. The residue was purified with column chromatography in CH₂Cl₂ and MeOH (30:1) mixture to afford 2-(4′-fluoromethyl)phenylbenzo[b]thiophene (125 mg, 0.5 mmol) as a brownish solid in a yield of 50%. 1H NMR (300 MHz, CDCl₃): δ 5.40 (s, 1H, —CH₂—), 5.56 (s, 1H, —CH₂—), 7.33 (t, 2H), 7.39 (d, 2H, J=7.8 Hz), 7.55 (s, 1H), 7.70 (d, 2H, J=7.8 Hz), 7.72-7.84 (m, 2H).

### EXAMPLE 4

### Synthesis of 2-(4′-fluoromethyl)phenylbenzo[b]furan

To the solution of (2-hydroxyphenyl)methyltriphenylphosphonim bromide (251 mg, 0.5 mmol) in toluene (10 mL), 4-(fluoromethyl)-1-benzenecarbonyl chloride (60 mg, 0.5 mmol) and Et₃N (150 μL, 1.3 mmol) were added. After refluxing for 10 hrs, the reaction mixture was filtered, and the filtrate volume was reduced by evaporating. The residue was purified with column chromatography in CH₂Cl₂ and MeOH (30:1) mixture to afford 2-(4′-fluoromethyl)phenylbenzo[b]furan (25 mg, 0.1 mmol) as a yellowish solid in a yield of 20%. 1H NMR (300 MHz, CDCl₃): δ 5.41 (s, 1H, —CH₂—), 5.58 (s, 1H, —CH₂—), 7.35 (t, 2H), 7.40 (d, 2H, J=7.8 Hz), 7.55 (s, 1H), 7.75 (d, 2H, J=7.8 Hz), 7.77-7.95 (m, 2H).

### EXAMPLE 5

### Synthesis of 2-[4′-(toluenesulfonyloxy)methyl]phenyl-1,3-benzothiazole

To the solution of 2-(4′-hydroxymethyl)phenyl-1,3-benzothiazole (50 mg, 0.9 mmol) in THF (20 mL), potassium hydroxide (350 mg, 9.0 mmol) and p-toluenesulfonyl chloride (135 mg, 2.7 mmol) were added. After stirring for 20 hrs at room temperature, the reaction mixture was filtered and the filtrate was evaporated and extracted with CH₂Cl₂ (10 mL×3). The organic layer was dried with Na₂SO₄ and evaporated in vacuo. The residue was purified with column chromatography in CH₂Cl₂ and MeOH (30:1) mixture to afford 2-[4′-(toluenesulfonyloxy)methyl]phenyl-1,3-benzothiazole (35 mg, 0.45 mmol) as a yellowish solid in a yield of 52%. ¹H NMR (300 MHz, CDCl₃): δ 3.65 (s, 3H, —CH₃), 5.45 (s, 2H, —CH₂—), 7.25 (d, 2H), 7.39 (m, 1H), 7.48-7.50 (m, 3H), 7.77 (d, 2H), 7.91 (d, 1H, J=7.8 Hz), 8.07-8.10 (m, 3H,).

### EXAMPLE 6

### Synthesis of 2-[4′-(toluenesulfonyloxy)methyl]phenyl-1,3-benzoxazole

To the solution of 2-(4′-hydroxymethyl)phenyl-1,3-benzoxazole (30 mg, 0.6 mmol) in THF (20 mL), potassium hydroxide (207 mg, 6.0 mmol) and p-toluenesulfonyl chloride (58 mg, 1.8 mmol) were added. After stirring for 20 hrs at room temperature, the reaction mixture was filtered and the filtrate was evaporated and extracted with CH₂Cl₂ (10 mL×3). The organic layer was dried with Na₂SO₄ and evaporated in vacuo. The residue was purified with column chromatography in CH₂Cl₂ and MeOH (30:1) mixture to afford 2-[4′-(toluenesulfonyloxy)methyl]phenyl-1,3-benzoxazole (20 mg, 0.3 mmol) as a yellowish solid in a yield of 52%. ¹H NMR (300 MHz, CDCl₃): δ 3.30 (s, 3H, —CH₃), 5.56 (s, 2H, —CH₂—), 7.25 (d, 2H, J=7.9 Hz), 7.37 (m, 2H), 7.53 (d, 2H, J=7.8 Hz), 7.58 (m, 1H), 7.75 (d, 2H, J=7.8 Hz), 7.78 (m, 1H), 8.29 (d, 2H, J=7.8 Hz).

### EXAMPLE 7

### Synthesis of 2-[4′-(toluenesulfonyloxy)methyl]phenylbenzo[b]furan

To the solution of 2-(4′-hydroxymethyl)phenylbenzo[b]furan (30 mg, 0.6 mmol) in THF (20 mL), potassium hydroxide (207 mg, 6.0 mmol) and p-toluenesulfonyl chloride (58 mg, 1.8 mmol) were added. After stirring for 20 hrs at room temperature, the reaction mixture was filtered and the filtrate was evaporated and extracted with CH₂Cl₂ (10 mL×3). The organic layer was dried with Na₂SO₄ and evaporated in vacuo. The residue was purified with column chromatography in CH₂Cl₂ and MeOH (30:1) mixture to afford 2-[4′-(toluenesulfonyloxy)methyl]phenylbenzo[b]furan (11 mg, 0.2 mmol) as a yellowish solid in a yield of 32%. ¹H NMR (300 MHz, CDCl₃): δ 3.05 (s, 3H, —CH₃), 5.40 (s, 2H, —CH₂—), 7.25 (d, 2H, J=7.9 Hz), 7.37 (m, 2H), 7.53 (d, 2H, J=7.8 Hz), 7.58 (m, 1H), 7.75 (d, 2H, J=7.8 Hz), 7.78 (m, 1H), 8.29 (d, 2H, J=7.8 Hz).

### EXAMPLE 8

### Synthesis of 2-[4′-(methanesulfonyloxy)methyl]phenyl-1,3-benzothiazole

To the solution of 2-(4′-hydroxymethyl)phenyl-1,3-benzothiazole (47 mg, 0.2 mmol) in CH₂Cl₂ (20 mL), TEA (278 μL, 2.0 mmol) and methanesulfonyl chloride (77 μL, 1.0 mmol) were added at 0 □. After stirring for 2 hrs at room temperature, the reaction mixture was evaporated and extracted with CH₂Cl₂ (10 mL×3). The organic layer was dried with Na2SO4 and evaporated in vacuo. The residue was purified with column chromatography in CH₂Cl₂ and MeOH (30:1) mixture to afford 2-[4′-(Methanesulfonyloxy)methyl]phenyl-1,3-benzothiazole (19 mg, 0.1 mmol) as a yellowish solid in a yield of 45%. ¹H NMR (300 MHz, CDCl₃): δ 2.98 (s, 3H, —CH₃), 5.31 (s, 2H, —CH₂—), 7.39-7.57 (m, 4H), 7.93 (d, 1H), 8.07-8.16 (m, 3H).

### EXAMPLE 9

### Synthesis of 2-[4′-(methanesulfonyloxy)methyl]phenylbenzo[b]thiophene

To the solution of 2-(4′-hydroxymethyl)phenylbenzo[b]thiophene (45 mg, 0.2 mmol) in CH₂Cl₂ (20 mL), TEA (263 μL, 1.9 mmol) and methanesulfonyl chloride (73 μL, 0.9 mmol) were added at 0□. After stirring for 2 hrs at room temperature, the reaction mixture was evaporated and extracted with CH₂Cl₂ (10 mL×3). The organic layer was dried with Na₂SO₄ and evaporated in vacuo. The residue was purified with column chromatography in CH₂Cl₂ and MeOH (30:1) mixture to afford 2-[4′-(methanesulfonyloxy)methyl]phenylbenzo[b]thiophene (9 mg, 0.05 mmol) as a yellowish solid in a yield of 21%. 1H NMR (300 MHz, CDCl₃): δ 3.42 (s, 3H, —CH₃), 4.50 (s, 2H, —CH₂—), 7.31-7.41 (m, 3H), 7.55 (s, 1H), 7.69-7.84 (m, 4H).

### EXAMPLE 10

### Synthesis of 2-(4′-[¹⁸F]fluoromethyl)phenyl-1,3-benzothiazole

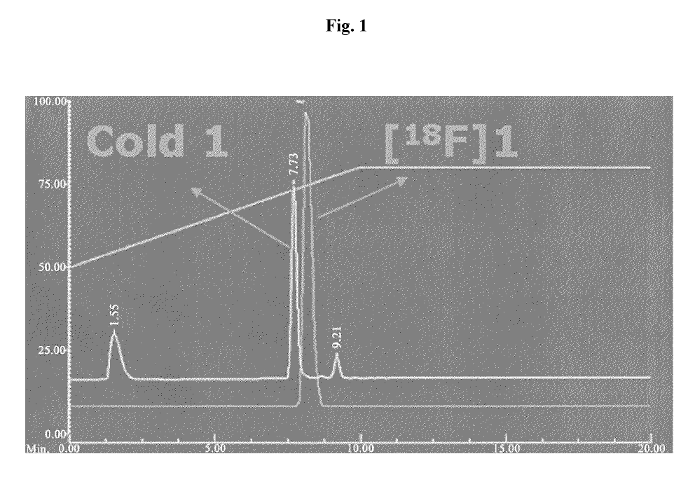

After bombardment of ¹⁸O⁻ water with proton beam accelerated by cyclotron, ¹⁸F⁻ was captured by passing through a QMA SepPak cartridge. ¹⁸F⁻ was eluted from the cartridge using 1 mL of 2.3% tetrabutyl ammonium bicarbonate in 83.8% acetonitrile/water solution. The eluate was evaporated by blowing argon with heating to 95˜100° C. 5 mg of 2-[4′-(toluenesulfonyloxy)methyl]phenyl-1,3-benzothiazole obtained from Example 5 in 3 mL acetonitrile was added and reacted for 30 min at 80° C. After drying by blowing with argon gas, the reaction mixture was purified by HPLC (XTerra C18 column, 7.5×30 mm, EtOH in water gradient: 50˜100%, 0˜10 min). As shown in FIG. 1, the labeling yield represented as 53% and radiochemical purity after purification was above 99%.

<Experiment 1> In Vitro Binding Assay

Aβ₁₋₄₀ or Aβ₁₋₄₂ peptides were incubated in phosphate buffer (pH=7.2) overnight to prepare precipitates as reported in literatures, and then aliquoted and stored at −70□. (Klunk, W. E.; Wang, Y.; Huang, G-F.; Debnath, M. L.; Holt D. P.; Mathis, C. A. Uncharged thioflavin-T derivatives bind to amyloid-beta protein with high affinity and readily enter the brain. Life Sci. 2001, 69(13), 1471-1914; Zhuang, Z.-P.; Kung, M.-P.; Wilson, A.; Lee, C.-W.; Plossl, K.; Hou, C.; Holtzman, D. M.; Kung, H. F.; Structure-activity relationship of imidazo[1,2-a]pyridines as ligands for detecting β-amyloid plaques in the brain. J. Med. Chem. 2003; 46(2), 237-243)

The binding affinities of benzothiophene derivatives to Aβ aggregates were evaluated by competitive binding assays using 3′-[¹²⁵I]I-BTA-1 as a radioligand. 100 μL of Aβ aggregates (20 nM in the final mixture), 100 μL of heterocyclic indene derivatives (10⁻⁶-10⁻¹² M in 50% ethanol containing 1 mM EDTA), 100 μL of 3′-[¹²⁵I]I-BTA-1 in 50% ethanol (0.1 nM in the final mixture) and 700 μL of phosphate buffered saline (pH 7.2) were added to test tubes and incubated for 3 h at room temperature for binding assay. Nonspecific binding was determined by incubation in the presence of 10 μM thioflavin-T. The reaction mixtures were filtered through Whatman GF/B glass filters and washed twice with 3 mL of 10% ethanol aliquots. Filters were counted using a Nat well counter. Kᵢ values of the benzothiophene derivatives were then calculated from the results and represented on Table 1.

TABLE 1
Ki values of heterocyclic indene derivatives
obtained from above Experiment 1.

Ki (nM)

| Compound | Aβ₁₋₄₀ | Aβ₁₋₄₂ |
|---|---|---|
| Benzothiazole | 26.91 ± 5.04 | 28.08 ± 4.44 |
| Benzoxaxole | 75.66 ± 43.24 | 59.87 ± 5.03 |

Above results show that Ki values to Aβ₁₋₄₀ and Aβ₁₋₄₂ did not show significant differences. Benzothiazole derivatives showed higher affinities than benzoxazole derivatives.

<Experiment 2> Biodistribution Experiment in Mice

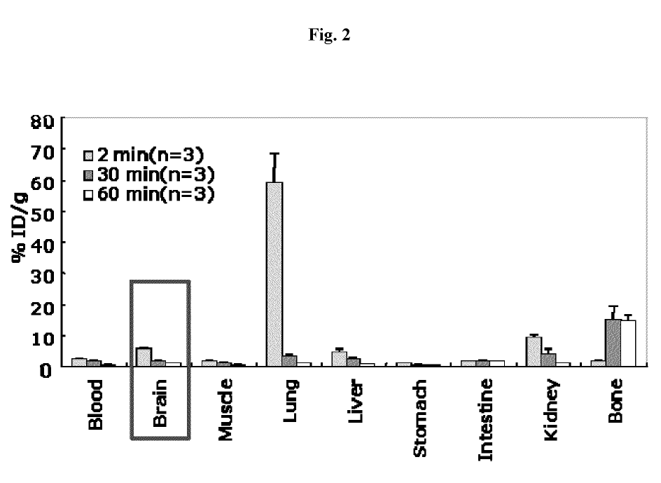

Male ICR mice (n=4/group, 29.9±1.6 g) were injected with 148 kBq/0.1 mL of 2-(4′-[¹⁸F]fluoromethyl)phenyl-1,3-benzothiazole through the tail vein. These injected mice were sacrificed by decapitation 2, 30 and 60 min post-injection. Blood, muscle, lung, liver, spleen, stomach, intestine, brain, and bone were rapidly separated, weighed and counted using a NaI well counter. Results are expressed as percentages of the injected dose per gram of tissue (% ID/g), and represented in FIG. 2. Brain uptake at min was 5.62±0.28% ID/g and ratio to 30 min was 3.75±0.97, which represent rapid uptake and wash out to and from normal brain. High bone uptake represents in vivo defluorination which exerts rapid decrease of background radioactivity.

<Comparative Experiment 1> Mice Biodistribution Study of ¹⁸F Labeled Benzothiophene Derivatives.

A comparative mice biodistribution study using 2-(4′-O-(2′-[¹⁸F]Fluoroethyl)hydroxyphenyl)benzothiophene and 2-(4′-O-(3′-[¹⁸F]Fluoropropyl)hydroxyphenyl) benzothiophene was performed according to the same procedure with above Experiment 2. The results are described in Table 2. Brain uptakes at 2 min and especially brain uptake ratio at 2 min and 30 min were significantly lower than the compounds in Experiment 2. The results show that the compounds of the present invention are better for β-amyloid agents than the compared compounds. Low bone uptakes represent high in vivo stability and thus showed long retention in the body due to low bone uptake and slow excretion.

TABLE 2
Mice biodistribution of ¹⁸F labeled benzothiophene derivatives
used in the above Comparative experiment 1.

| | | |
|---|---|---|
| Tissue | 2 min | 30 min |

2-(4′-O-(2′-[¹⁸F]Fluoroethyl)hydroxyphenyl)benzothiophene

| | | |
|---|---|---|
| Blood | 3.5 ± 0.2 | 3.8 ± 0.4 |
| Muscle | 2.9 ± 0.3 | 2.7 ± 0.3 |
| Fat | 0.9 ± 0.3 | 2.1 ± 1.0 |
| Heart | 8.8 ± 1.0 | 3.2 ± 0.2 |
| Lung | 9.3 ± 1.1 | 3.4 ± 0.6 |
| Liver | 9.3 ± 1.5 | 3.9 ± 0.7 |
| Spleen | 4.4 ± 0.8 | 2.7 ± 0.5 |
| Stomach | 2.1 ± 0.7 | 2.5 ± 0.3 |
| Intestine | 2.7 ± 0.2 | 7.2 ± 0.6 |
| Kidney | 11.8 ± 0.9 | 4.1 ± 0.7 |
| Brain | 5.2 ± 0.4 | 5.2 ± 0.5 |
| Bone | 2.5 ± 0.2 | 3.3 ± 0.2 |

2-(4′-O-(3′-[¹⁸F]Fluoropropyl)hydroxyphenyl)benzothiophene

| | | |
|---|---|---|
| Blood | 3.3 ± 0.5 | 1.4 ± 0.1 |
| Muscle | 3.0 ± 0.2 | 1.6 ± 0.1 |
| Fat | 0.7 ± 0.1 | 1.9 ± 0.4 |
| Heart | 11.9 ± 0.6 | 1.8 ± 0.3 |
| Lung | 10.2 ± 1.4 | 2.2 ± 0.6 |
| Liver | 17.0 ± 0.6 | 6.1 ± 0.6 |
| Spleen | 4.1 ± 1.2 | 1.5 ± 0.2 |
| Stomach | 1.8 ± 0.5 | 1.0 ± 0.4 |
| Intestine | 2.4 ± 0.2 | 5.5 ± 0.4 |
| Kidney | 13.5 ± 1.2 | 4.4 ± 0.4 |
| Brain | 3.3 ± 0.2 | 4.0 ± 0.4 |
| Bone | 2.2 ± 0.2 | 10.7 ± 0.6 |

### Advantageous Effect

As described above, heterocyclic indene derivatives of the present invention can be easily labeled with radioisotopes. And the heterocyclic indene derivatives and their radiolabeled compounds of the present invention have excellent features for β-amyloid deposit imaging such as high affinity to β-amyloid plaques, high initial brain uptake and rapid clearance from the brain due to high BBB permeability.

The compounds of the present invention can be used for imaging β-amyloid plaques by binding to the deposited β-amyloid plaques in the brain of Alzheimer's disease patients. And these compounds also can be used for diagnosis, prevention or therapy of Alzheimer's disease caused by of β-amyloid plaques.

What is claimed is:

1. A heterocyclic indene derivative compound represented by the following Formula 1, wherein X is selected from a group consisting of O, S and NH; Y is CH or N; Z is selected from a group consisting of F, Cl, Br, I and a sulfonyl derivative; and R₁ to R₈ are independently selected from a group consisting of hydrogen, C₁˜C₄ alkyl, F, Cl, Br, I, C—CH₃, C—CH₂—CH₃, C—CH₂—CH₂—CH₃ and OH, respectively.

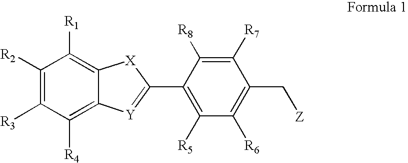

2. The compound of claim 1 wherein R₁ to R₈ are hydrogen, respectively.

3. The compound of claim 1 wherein the sulfonyl derivative is selected from a group consisting of methansulfonyl (OMs), toluenesulfonyl (OTs), trifluoromehanesulfonyl (OTf), benzenesulfonyl, nitrobenzenesulfonyl, mesitylenesulfonyl, triisopropylbenzenesulfonyl, chlorobenzenesulfonyl and dichlorobenzenesulfonyl.

4. A radioisotope labeled compound of the heterocyclic indene derivative compound of claim 1, which has the following Formula 2, wherein X is selected from a group consisting of O, S and NH; Y is CH or N; and R₁ to R₈ are independently selected from a group consisting of hydrogen, C₁˜C₄ alkyl, F, Cl, Br, I, C—CH₃, C—CH₂—CH₃, C—CH₂—CH₂—CH₃ and OH, respectively.

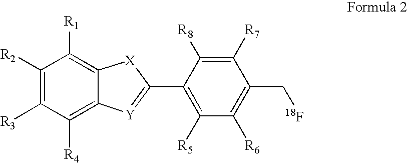

5. The compound of claim 4 wherein R₁ to R₈ are hydrogen, respectively.

6. A method for preparation of the compound of claim 4, which comprises reacting the compound of claim 1 with an activated ¹⁸F.

7. The method of claim 6, wherein the activated ¹⁸F is in form of a quaternary ammonium salt or a cation containing crown ether salt.

8. The method of claim 7, wherein the quaternary ammonium is tetrabutyl ammonium bicarbonate and the cation containing crown ether is a mixture of K₂CO₃ and Kryptofix 2.2.2.

9. A composition for β-amyloid imaging comprising the radioisotope labeled compound of claim 4.

10. The composition of claim 9, wherein the radioisotope labeled compound is contained in an amount of a range from 0.1 mCi to 10 Ci in the composition at just before use.

11. An injectable pharmaceutical composition for diagnosis of Alzheimer's disease comprising the radioisotope labeled compound of claim 4.

12. The injectable pharmaceutical composition of claim 11, wherein the radioisotope labeled compound is contained in an amount of a range from 0.1 mCi to 10 Ci in the composition at just before use.
